(19) United States
(12) Patent Application Publication
(10) Pub. No.: US 2006/0111681 A1
(43) Pub. Date: May 25, 2006

(54) Free-standing urine collection bag and urine bag system

(76) Inventor: Robert D. Vernon, Denver, CO (US)

Correspondence Address: THE HARRIS FIRM, 2830 SOUTH MEADE, ARLINGTON, VA, 22206, US

(21) Appl. No.: 11/263,660

(22) Filed: Nov. 1, 2005

Related U.S. Application Data
(60) Provisional application No. 60/625,921, filed on Nov. 8, 2004.

(51) Int. Cl. A61M 1/00; A61F 5/44

(52) U.S. Cl. 604/326; 604/331

(57) ABSTRACT

A self-standing urine collection bag that includes a bag of flexible material having an air and liquid-tight closure; a pair of opposing substantially flat, flexible side panels that are bonded to one-another and that expand when the bag is filled; an inwardly-recessed, gusseted bottom panel that is folded when the bag is empty, and unfolded when the bag is full; wherein the bottom panel provides support for the side panels so that, when filled with urine, the bag is self-supporting and can maintain a stable vertical standing position when resting on a substantially horizontal surface. A self standing urine bag system that maintains the bag's volumetric reading precision by using a reusable, rigid frame to deploy fully the flexible, disposable bag.

### FIELD OF THE INVENTION

The present invention relates to a free-standing urine collection bag, and more particularly, to a free-standing disposable urine collection bag that allows easy measurement of urine, convenient handling and disposal of urine, and has a low cost of manufacturing.

### BACKGROUND OF THE INVENTION

Conventional hospital urine collection bags, which typically hang at the foot of a patient's bed, present a number of drawbacks related to the measurement, handling and disposal of urine. These bags do not provide an accurate measure of urine volume contained within them and must be emptied into a separate graduated cylinder for purposes of measurement. They also have little structural rigidity, which makes them hard to handle. These bags also frequently empty from their bottoms, and they often cause urine to be released under pressure. This can lead to urine splashing healthcare workers while emptying urine into measuring cups. In addition, the outlet tubes and clamps used to keep urine within such bags can easily fail resulting in urine being released onto the floor of the hospital room.

U.S. Pat. No. 6,345,911 discloses a lock top canister bag and method of manufacture, which includes an inwardly folded gusset at its lower end which can be flattened to expand and flatten the lower end of the bag so that it can stand in an open condition. However, this bag does not provide the high degree of support or stability required to self-maintain a vertical standing position when filled with human urine, nor does it provide a single vessel for collection, storage, accurate measurement, and disposal of urine.

Hence, the prior art fails to provide a urine collection bag that provides adequate support or stability for self-maintenance of a vertical standing position when filled with human urine, nor does it provide a single vessel for collection, storage, accurate measurement, and disposal of urine.

### SUMMARY OF THE INVENTION

Thus, the present invention is directed to a urine collection bag or urine collection bag system that provides adequate support and stability for self-maintenance of a vertical standing position before, during, and after filling with human urine.

The present invention is also directed to a urine collection bag or urine collection bag system that provides a single vessel for collection, storage, accurate measurement, and disposal of urine.

One aspect of the present invention is directed to a pair of opposing, substantially flat, flexible side panels that are bonded to one-another to form an enclosed body, and that move apart when the urine bag is filled with gas or urine. The bag also has an inwardly-recessed, gusseted bottom panel that is folded when the urine bag is empty, and unfolded when the urine bag is full. The bottom panel is bonded within the body to form an air and liquid-tight closure, and the bottom panel provides support for the side panels so that, when filled with urine, the urine bag is self-supporting and can maintain a stable vertical standing position when resting on a substantially horizontal surface.

In another aspect, the bottom panel is fully deployed by a force internal to the bag, which provides accurate and precise volumetric readings of urine while urine is in the bag.

In still another aspect, the bag has volumetric measuring indicia on at least one of the side panels.

In yet another aspect, the body is a uniform structure which includes an integrated pour handle and integrated hanging loop.

In still another aspect, the deployment is maintained by a rigid external insert that operably fits into a recess in the bag.

In yet another aspect, the insert is a side panel separator that comprises hard foam, a rigid frame, or plastic.

In still another aspect, the bag has an elastically expanding, collapsible insert that separates the side panels and deploys the gusseted bottom when the bag is empty of liquid.

In yet another aspect, the insert collapses during vacuum packaging of the bag so that the bag flattens, and expands when the bag is unpacked or opened for use.

In still another aspect, the insert is made from reticulated foam or a rigid plastic frame.

In yet another aspect, the bottom panel is fully and operatively deployed by positive gas pressure internal to the bag that is greater than an ambient pressure exerted on the outside of the bag.

In still another aspect, the bag is deployed by manual inflation through a blow tube inlet, or by a release of gas from a separate, compressed gas chamber.

In yet another aspect, the bag has dual purpose liquid inlet and outlet fittings that allow air to escape when urine enters the bag, and allow air to enter when urine exits the bag.

In still another aspect, at least one of the fittings includes activated carbon to adsorb odors from air vented out of the bag during its filling with urine.

In yet another aspect, the bag has a handle that is made from at least one of the side panels.

Another aspect of the invention is directed to a self-standing urine collection bag, comprising a bag of flexible material having a top and a bottom portion, the outer surfaces of which are operatively and releasably coupled to respective upper and lower ends of a rigid external frame. The rigid frame provides support for the bag so that, when filled with urine, the bag can maintain a stable vertical standing position when resting on a substantially horizontal surface.

In another aspect the frame operably places the bag in tension across multiple internal distances so that a measurement of volume of urine in the bag, using measuring indicia on the bag or frame, substantially and reproducibly conforms to an accurate volume of urine.

In still another aspect, the tension placed upon the bag is three-dimensional.

In yet another aspect, the frame has a telescoping arm, the adjustable movement of which imparts the tension.

In still another aspect, the bag has volumetric measuring indicia printed or embossed on one of the side panels.

In yet another aspect, the bag operatively has a handle that is made from the rigid frame.

Another aspect of the invention is directed to a self-standing urine collection bag system, comprising a flexible, side portion that is formed of one or more plastic sheet panels bonded to one another along their sides and tops to form a body, and that expands as the urine collection bag is filled. The bag system also includes an inwardly-recessed, bottom panel or plug that is bonded to an internal wall of the side portion to form a supportive interface. The supportive interface provides support for the side portion so that, when filled with urine, the urine collection bag is self-supporting and can maintain a stable vertical standing position when resting on a substantially horizontal surface. The supportive interface provides regular and full deployment of the bag for accurate measurement of urine while urine is in the bag, by measuring indicia on the bag.

In another aspect the bag has dual purpose liquid inlet and outlet fittings that allow air to escape when urine enters the bag, and allow air to enter when urine exits the bag.

In still another aspect, the urine bag system further comprises a frame that operably places the bag in tension across multiple internal distances so that a measurement of volume of urine in the bag, using measuring indicia on the bag, substantially and reproducibly conforms to an accurate volume of urine.

In yet another aspect, at least one of the fittings includes activated carbon to adsorb odors from air vented out of the bag during filling with urine.

In still another aspect, the bag has a handle that is made from a cylindrical side portion.

In yet another aspect, the bag is made from a cylindrical body, and at least one embossing structure on the side portion provides vertical support for the bag.

In still another aspect, a rod runs from the top of the cylindrical portion to the bottom panel or plug to provide additional vertical support, and stability while emptying the bag.

In yet another aspect, the inlet and outlet fittings are sealed between the two sheet panels.

### BRIEF DESCRIPTION OF THE DRAWINGS

In the accompanying drawings, which form a part of the specification and are to be read in conjunction therewith, and in which like reference numerals are used to indicate like parts in the various views:

FIG. 1 is a front perspective view of a urine collection bag according to the present invention;

FIG. 1a is a cutaway front perspective view of another embodiment of the bag of FIG. 1;

FIG. 2 is a side view of the bag of FIG. 1;

FIG. 3 is a bottom perspective view of the bag of FIG. 1;

FIG. 3a is a bottom perspective, assembly view of the bag in FIG. 1 and a form-fitting, deployed-gusset, bottom insert according to the present invention;

FIG. 3b is a bottom perspective, assembly view of the bag in FIG. 1 and another embodiment of a form-fitting, deployed-gusset, bottom insert according to the present invention;

FIG. 3c is a bottom perspective, assembly view of the bag in FIG. 1 and another embodiment of a form-fitting, deployed-gusset, bottom insert according to the present invention;

FIG. 4 is a bottom perspective, assembly view of the bag of FIG. 1 that includes a collapsing and elastically expanding insert according to the present invention;

FIG. 5 is a side cross-sectional view of the bag and insert of FIG. 4 before vacuum sealing, along line B-B;

FIG. 6 is a side cross-sectional view of the bag and insert of FIG. 4 after vacuum sealing, along line B-B;

FIG. 7 is a side cross-sectional view of the bag and insert of FIG. 4 after self-inflation, along line B-B;

FIG. 8 is another embodiment of a collapsing and expanding insert according to the present invention;

FIG. 9 is a front perspective, assembly view of another embodiment of a urine collection bag according to the present invention;

FIG. 10 is a front perspective view of the bag of FIG. 9 when being used to receive urine;

FIG. 11 is an assembly perspective view of a urine bag, as seen from beneath the bag, and a frame, as seen from above the frame, according to the present invention;

FIG. 12 is a front perspective view of the bag and frame of FIG. 11 when being used to receive urine;

FIG. 13 is a front perspective, assembly view of another embodiment of a urine bag according to the present invention, which has a hang tab fitting, a connector fitting, and a split cap;

FIG. 14 is a top plan view of the split cap of FIG. 13;

FIG. 15 is a top plan view of the connector fitting of FIG. 13;

FIG. 16 is a top assembly view of the bag of FIG. 13 and a frame according to the present invention;

FIG. 17 is a front plan view of the bag and frame of FIG. 16;

FIG. 18 is a side plan view of the bag and frame of FIG. 16;

FIG. 19 is a front assembly view of another embodiment of a urine bag and frame according to the present invention, wherein the frame has a telescoping L-shaped arm and base;

FIG. 20 is a side plan view of the adjustable frame of FIG. 19;

FIG. 21 is a top plan view of the L-shaped arm of FIG. 19; and

FIG. 22 is a top plan view of the base of FIG. 19.

### DETAILED DESCRIPTION OF THE INVENTION

As illustrated in the accompanying drawings and discussed in detail below, one aspect of the present invention is directed to a free-standing disposable urine collection bag that allows easy measurement of urine, convenient handling and disposal of urine, and has a low cost of manufacturing. In particular, this aspect provides full, reproducible, and continuous deployment of the internal area of the bag, which allows accurate and precise volumetric readings of urine while the urine is still in the bag. This aspect then allows quick, safe and easy storage and disposal of urine, directly from the bag.

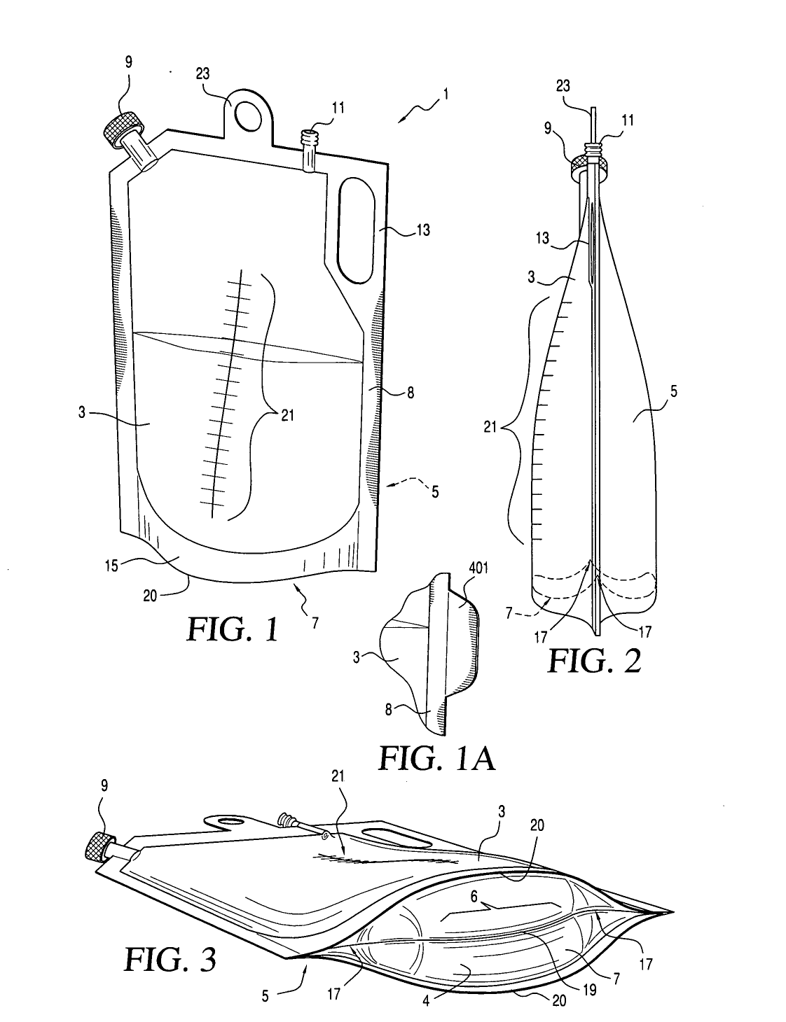

In one embodiment, bag deployment occurs as a result of forces exerted from inside the bag. Referring to FIGS. 1, 2 and 3, disposable urine bag 1 includes front 3 and back 5 flexible plastic side panels, and inwardly-recessed, gusseted, bottom panel 7, which are attached to one another according to one of a variety of methods. Several such methods, all of which shall be deemed as applicable for use to attach various panels and fittings one to another in each of the embodiments described below, include, but are not limited to, heat welding, heat sealing, heat bonding, and other suitable permanent attachment methods. Several such methods for processes used to make bags suitable for this aspect of the invention are described in U.S. Pat. No. 5,971,613, which is hereby incorporated by reference in its entirety.

Panels 3 and 5 are mirror images of each other, and they come together to form the uniform body structure of bag 1, which includes integrated pour handle 13 and integrated hanging loop 23. Panel 7 is a single elongated panel that is inwardly-recessed into the body of bag 1. In one embodiment, bottom panel 7 has either rounded (not shown) or pointed ends 17, although other shapes are suitable for use with this aspect of the invention. When bag 1 is empty, middle portion 6 is the same, or nearly the same, distance from bottom edges 20 as ends 17. When bag 1 is full, however, ends 17 are inwardly-recessed within bag 1, i.e., away from bottom edges 20, to a greater extent than middle portion 6, which bulges out towards edges 20.

This bulging and flattening of middle portion 6 occurs in response to the filling and emptying of bag 1. In particular, bottom panel 17 responds to the emptying and filling of bag 1 by folding and unfolding. When bag 1 is empty and flat, panel 7 is folded onto itself along lengthwise crease 19 so that bottom surfaces 2 and 4 meet each other. As the volume of liquid or gas that bag 1 holds decreases, side panels 3 and 5 come together. Panels 3 and 5 thereby force panel 7 to fold onto itself along crease 19. As a result, crease 19 is pushed further into bag 1, i.e., away from bottom edges 20 of bag 1. When bag 1 is filled with liquid or gas, side panels 3 and 5 are forced apart, which deploys bottom panel 7. Panel 7 deploys by unfolding along crease 19, which moves outwardly, i.e., toward bottom edges 20 of bag 1.

Thus, under the pressure or weight of incoming or expanding gas or liquid, inwardly-recessed, gusseted bottom panel 7 unfolds at bag 1's lower end to lend stability to bag 1. The force of gas or liquid against the interior surface of bottom panel 7, and the inner surfaces of side panels 3 and 5, separates and provides support for the bottom portions of sides 3 and 5.

Under the more particular weight of urine that collects at the bottom of bag 1, panel 7 bulges downward and panels 3 and 5 separate, thereby providing stability for urine bag 1 to stand upright on its own. Under a sufficient weight of urine the separation of panels 3 and 5, which coincides with the deployment of panel 7, thereby allows bag 1 to stand by itself on a counter or other substantially horizontal surface.

The deployment of bottom panel 7 thus provides immediate and concurrent volumetric reading as bag 1 is filled with urine, without the use of additional reading instruments. Volumetric measuring indicia 21 are embossed or printed onto front panel 3 to allow for accurate volumetric measuring of urine without having to empty bag 1 into a separate measuring device. Used in tandem with the self-supporting feature of bag 1, an accurate measurement of bag 1's contents can be determined simply by visual examination of bag 1 immediately after, or concurrent with, its filling. This feature eliminates the need to empty bag 1 into a graduated cylinder or other measuring container.

Panels 3, 5, and 7 are made from transparent or translucent plastic, but they can be made of any lightweight flexible material suitable for containing biological fluids. Lightweight plastic polyolefin panels 3, 5, and 7 are highly flexible, but can support a sizable weight of urine, at least as great as a typical large adult human bladder discharge. Sides 3, 5, and 7 buckle as bag 1 is filled, but uniformly thick bottom portions 15 provide stability to support bag 1 when it is full.

Regarding the filling and emptying of bag 1, urine flows into bag 1 through fitting 11, and air flows into fitting 11 when bag 1 is emptied. Fitting 11 is a plastic valve, but it can be any valve suitable for performing the above-described dual functionality. Alternately, more than one fitting can be used to perform these two functions.

Bag 1 also has dual-purpose, urine outlet and air-escape valve fitting 9, through which (1) air or other gas exits bag 1 as urine fills bag 1, and (2) urine is poured out of bag 1 (to dispose of urine when bag 1 is full). Fitting 9 is a plastic screw close valve, but it can be any valve suitable for performing the above-described dual functionality. Alternately, more than one fitting can be used to perform these two functions.

In one embodiment, fitting 9 has a round opening, and an eye-shaped base about which panels 3 and 5 are bonded, which provides an air-tight and liquid-tight seal between bag 1 and fitting 9.

Fittings 9 and 11 are made of plastic, but they can be made from any synthetic or natural lightweight airtight material suitable for containing biological fluids. Fittings 9 and 11 are bonded between sides 3 and 5, but can be attached in any manner suitable to provide an airtight seal.

While collecting urine, bag 1 hangs on a bed frame, mobile stand, or elsewhere by integrated hanging loop 23. Once bag 1 is full, however, bag 1 is detached easily from its catheter tube, which is connected to fitting 11, and taken down either before or after its final volumetric urine measurement is recorded. During such final volumetric measurement, bag 1 can easily and safely stand on any flat surface until time permits for urine disposal out of fitting 9, or disposal of bag 1 and its contents altogether.

Referring to FIG. 1a, in one embodiment tab handle 401 is integrated into the body structure of bag 1 as part of, and along the middle (or bottom) of, right side strip 8. This placement of handle 401 nearer to the weight of urine-filled bag 1, provides balance and stability while pouring urine out of bag 1.

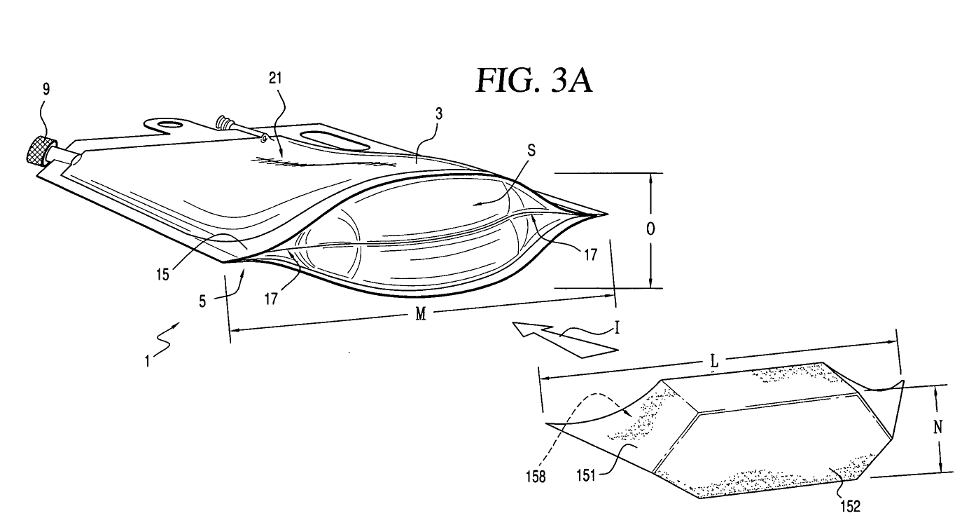

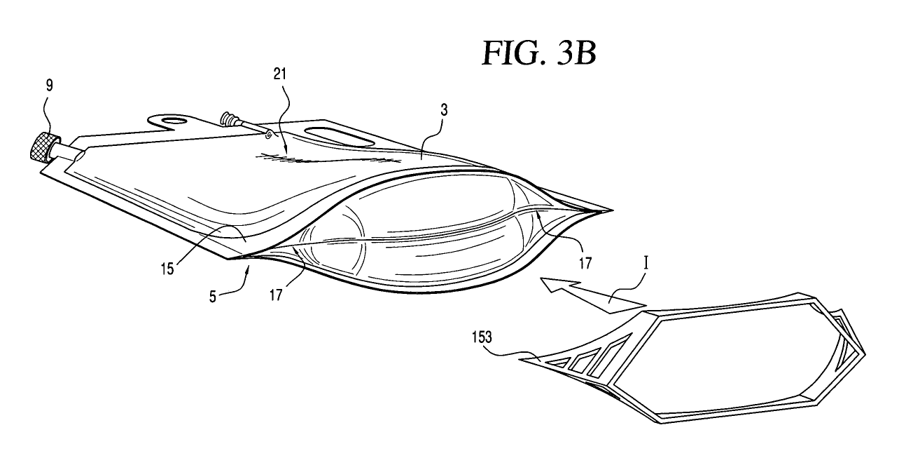

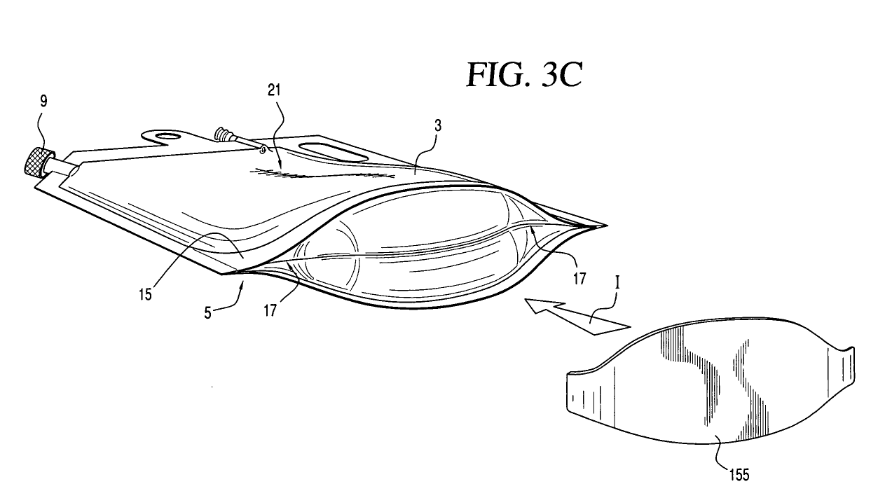

Referring to FIG. 3a, in one embodiment, solid, non-compressible, form-fitting, and reusable, Styrofoam® bottom insert 151 ensures full, reproducible deployment of panel 7. Insert 151 is molded to have the same dimensions as space S, which is defined by the inside of bag 1's bottom portions 15 and panel 7, when panel 7 is fully deployed. Thus, length L of insert 151 is the same as length M of space S, and width N of insert 151 is the same as width O of space S. Also, top face 158 form-fits and cradles the external, fully deployed surface of panel 7.

Insertion of insert 151 into the bottom of bag 1 in accordance with direction arrow 1, achieves and maintains full and reproducible deployment of bottom panel 7, which provides accurate and precise volumetric reading without the use of additional reading instruments. To the extent that panel 7 is not fully deployed, such as where an insufficient weight of urine fills bag 1, the volume measured by indicia 21 may be inaccurate or imprecise. This inaccuracy or imprecision is caused whenever panel 7 does not regularly and repeatedly deploy to the same degree under the same volume of urine or other liquid. Such variations in deployment are caused by many variables, including for example, atmospheric pressure, temperature, liquid density, filling flow rate and other variables. Insert 151, and the other inserts described herein below, however, ensure full, regular, reproducible, repeated, and continuous deployment of panel 7 by providing and maintaining the proper distances between the respective lengths of panels 3 and 5.

Insert 151 is attached to bag 1 using an adhesive or other suitable fastening or attachment device. Alternately, insert 151 is sized such that its dimensions makes it fit just tightly enough within panel 7 and bottom portions 15 to be retained within space S. In either case, insert 151 provides and maintains the proper distances between the respective lengths of panels 3 and 5, to allow accurate and precise volumetric urine measurement within bag 1.

Insert 151 also has flat bottom 152, which rests on the surface on which bag 1 sits. Flat bottom 152 thereby adds to, and maintains, the stability of bag 1.

Suitable materials for insert 151 include, but are not limited to, any solid foam that can be adhesively, or otherwise, attached or positioned between panels 3 and 5. These materials include, but are not limited to, any rigid natural or synthetic material such as wood, plastic, or foam. However, any sufficiently rigid material that will provide and maintain separation of panels 3 and 5 is suitable for insert 151.

Referring to FIG. 3b, in another embodiment, rigid and reusable hard plastic frame insert 153 ensures full, reproducible deployment of panel 7. When frame insert 153 is inserted into the bottom of bag 1 in accordance with direction arrow 1, the resulting full and reproducible deployment of bottom panel 7 provides accurate and precise volumetric reading without the use of additional reading instruments.

Frame insert 153 also has flat bottom 154, which rests on the surface on which bag 1 sits. Flat bottom 154 thereby adds to, and maintains, the stability of bag 1.

Suitable materials for frame insert 153 include any rigid material that can be adhesively, or otherwise, attached or positioned between panels 3 and 5. These materials include, but are not limited to, any rigid natural or synthetic material such as wood, plastic, or hard foam. However, any sufficiently rigid material that will provide and maintain separation of panels 3 and 5 is suitable for insert 153.

Referring to FIG. 3c, in another embodiment, rigid and reusable, hard plastic spacer insert 155 ensures full, reproducible deployment of panel 7. When insert 155 is inserted onto the bottom of bag 1 in accordance with direction arrow 1, insert 155 acts as a spacer that keeps panels 3 and 5 specified distances apart, along the length of bottom portion 15. Although insert 155 does not extend across the length of panel 7, it adequately maintains panels 5 and 3 apart from one another such that panel 7 is fully deployed. Insert 155 can alternately be shorter, or comprise a thin, rigid lattice. The resulting full and reproducible deployment of bottom panel 7 provides accurate and precise volumetric reading, without the use of additional reading instruments.

Suitable materials for spacer insert 155 include any hard plastic that can by adhesively, or otherwise, attached or positioned between panels 3 and 5 include any rigid, natural or synthetic material such as wood or plastic. However, any sufficiently rigid material that will provide and maintain separation of panels 3 and 5 is suitable for insert 155.

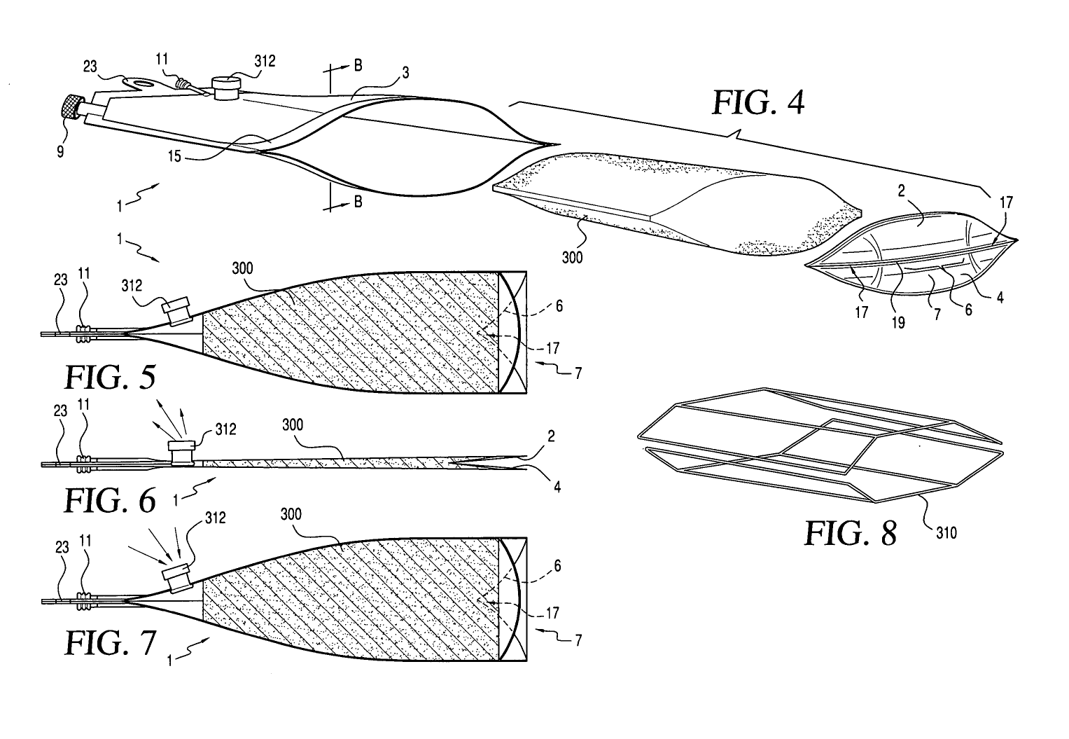

Referring to FIG. 4, in one embodiment collapsible and elastically expandable, reticulated foam insert 300 is placed into bag 1 prior to heat welding panel 7 to panels 3 and 5.

Referring to FIG. 5, when bag 1 is closed, foam insert 300 pushes panels 3 and 5 apart, and thereby fully and reproducibly deploys panel 7.

Referring to FIG. 6, when bag 1 is vacuum packed inside an outer sterile container (not shown), all of the air inside bag 1 is removed. As a result, foam insert collapses and panels 3 and 5 come together to form relatively flat bag 1. Bag 1 is stored and transported flat, and remains flat until use.

Referring to FIG. 7, just before use, bag 1 is removed from its sterile vacuum packing, at which point it self-inflates by the elastic expansion of foam insert 300, which causes buckling of panels 3, 5 and 7. Once inflated, bag 1 is ready for use and stable for self-supported upright placement on any substantially horizontal, flat surface.

In one embodiment, foam insert 300 is impregnated with an antimicrobial agent such as silver-laden zeolite. This antibacterial material discourages bacteria growth within bag 1 and accordingly the chance of patient infection by urine-borne pathogens. Suitable antimicrobial substances include, but are not limited to, cations, anions, non-ionic agents, enzymes, and sugar alcohols. More specifically, these agents include, but are not limited to, Cu²⁺, Zn²⁺, Sn²⁺, alexidine, chlorhexidine, cetyl pyridinium chloride, hexetidine, quaternary ammonium compounds, sanguinaria extracts, florides, triclosan, Listerine®, other phenolic compounds, sanitizers, hospital disinfectants, general use disinfectants, and sterilizers.

Referring to FIG. 8, in one embodiment, collapsible and elastically expanding rigid, plastic frame 310 is used in place of foam insert 300. Frame 310 can be made from any relatively rigid, synthetic or natural material that elastically returns itself to shape, or is otherwise spring loaded.

In one embodiment, bag 1 self-inflates after manual depression of a nitrogen or carbon-dioxide generator, such as is disclosed in U.S. Pat. Nos. 4,832,968 and 5,571,548, which are hereby incorporated by reference in their entireties. Other suitable internal, gas-generating chambers could be used, as long as they heat-sealed within bag 1, between bag 1's panels 3 and 5. Alternately, any other non-metal or metal, internal chamber, gas-generator is suitable for this aspect of the invention as well, however.

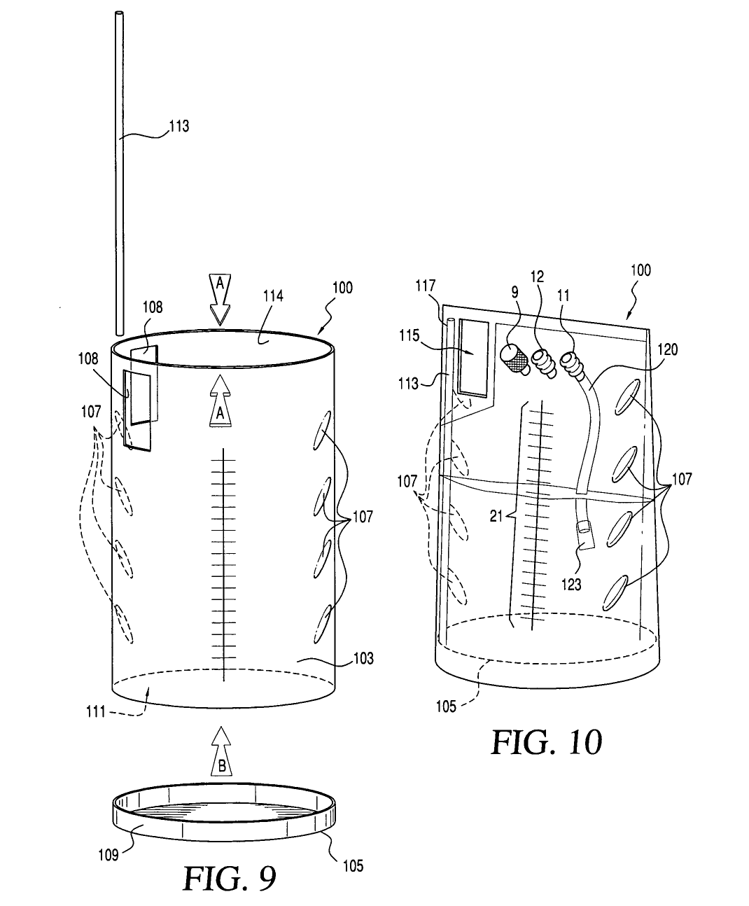

Referring to FIG. 9, in an alternate embodiment, self-standing urine collection bag 100 is composed primarily of cylindrical portion 103 and base portion 105. Cylindrical portion 103 is fabricated from plastic or a coated paper product. Embossed structures 107 in the paper or plastic material strengthen the material and increase its ability to make bag 100 self-supporting, particularly as bag 103 fills with urine. Suitable materials for cylindrical portion 103 and base portion 105 include polyolefin. However, any sealable and sufficiently rigid material that provides self-standing construction is suitable for this embodiment.

Base portion 105 is formed from a separate piece of material in the form of an inverted pan, which acts as a plug. Thus, rectangular, round, elliptical, or other shapes connected to and enclosed within interior walls 111 of portion 103 are suitable for use as plugs in this embodiment.

Interior side wall 111 of cylindrical portion 103 and exterior side wall 109 of base portion 105 are in contact with, and attached to, each other, with a suitable adhesive or other bonding method, such as heat welding. Thus, the area of their attachment becomes the primary support structure for bag 100. This embodiment thus eliminates the need for a separate insert required for the base of a standup pouch.

When stored empty, bag 100's base portion 105 folds substantially flat onto flattened cylindrical portion 103. When ready for use, bag 100 is unfolded such that the bottom of base 105 is set flat on a horizontal surface.

Referring to FIG. 10, cutout portion 115 of bag 100 provides hang tab 117, which allows a user to place bag 100 on a hook or other raised arm. Alternately, hang tab 117 is formed by heat welding diametrically opposed apertures 108, which when sealed together are centrally positioned at the top of bag 100.

Inserted grasp rod 113 runs from base 105 to the top of bag 100. Grasp rod 113 is heat welded between the sides of bag 100, and extends freely into bag 100. Grasp rod 113 can be held at any point along its length. This feature gives bag 100 handling stability while a user tilts it to empty it of urine.

Fittings 9 and 11 are heat welded into holes on wall 111 of cylinder 103 before (or after) walls 111 and 114 are attached to one another.

In an alternate embodiment, fittings 9 and 11 are bonded between walls 111 and 113 of cylinder 103 as these walls are heat sealed, heat bonded, heat welded, or otherwise attached to one another.

Drip tube 120 provides a controlled passage for urine to empty into bag 100, which thereby alleviates the dripping sound commonly associated with drip chamber urine collection bags. One-way flutter valve 123 at the end of tube 120 ensures that urine does not flow back into tube 120 or out of bag 100. Any one-way valve suitable for stopping backflow of urine may be used, however.

In one embodiment, the user blows air into one-way valve 12 to inflate bag 100 with air while momentarily closing escape valve fitting 9 with his or her hand. This manual inflation by the user converts bag 100 into a semi-rigid container, which helps ensure that volumes measured according to indicia 21 accurately correspond to the volume of liquid present in bag 100.

One additional, separate one-way inlet valve 12 is used for manual inflation of bag 100 but not used for later urine in-flow, so that bacteria from the inflating user's breath do not contaminate urine inflow tube 120. Use of valve 12 to prevents bacterial growth up tube 120, through fitting 11, and towards a patient's catheter, which reduces the likelihood of infection due to bacterial migration from whomever inflates bag 100 to the patient.

As seen, internally expanding, elastic solid structures and devices that provide an increase in internal air pressure within a urine bag, are included in, but do not limit, the types of devices that can be used as part of this aspect of the invention.

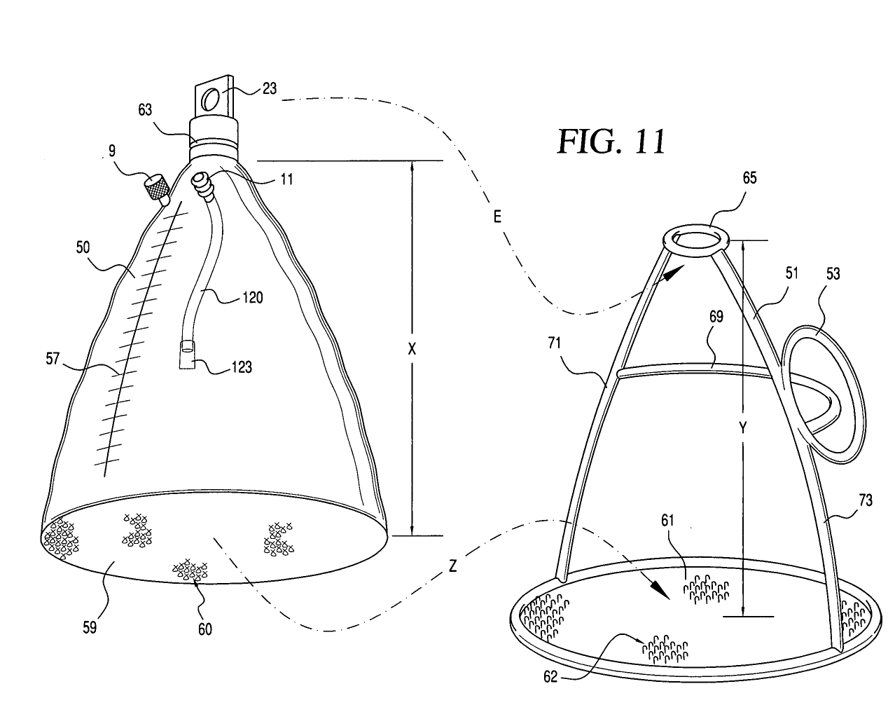

Other embodiments that enhance full bag deployment, and thus precise and accurate urine measurement, for example, work externally upon an un-deployed bag, as is described immediately herein below. Referring to FIG. 11, to the extent that urine bag 50 is not fully deployed, such as where an insufficient weight of urine fills bag 50, the volume measured by indicia 57 may be inaccurate. To the extent that bag 50 does not regularly and repeatedly deploy to the same degree under the same volume or weight of urine or other liquid, the volume measured by indicia 57 may be inaccurate or imprecise. The use of tensioning frame 51, however, ensures full, regular, reproducible, repeated, and continuous deployment of bag 50 by consistently and reproducibly maximizing, without undue stress, the distances between multiple opposing internal points within the flexible surface of disposable urine bag 50. Frame 51, like other frames described herein, also maintains these distances until accurate and precise volumetric readings have been taken. Frame 51, moreover, can be used repeatedly and hygienically, over-and-over-again, with numerous disposable bags.

Thus, collapsible plastic bag 50 fits within external frame 51. When fit inside of frame 51, bag 50 is tensioned such that it is vertically supported and allows accurate volumetric measurement of urine with indicia 57. Loop material 60, which is adhesively adhered to bottom 59 of bag 50, is releasably attached to hook material 62 on internal bottom 61 of frame 51 according to direction arrow Z. In addition, height X of bag 50 is approximately the same, or equal to, length Y on frame 51.

Plastic snap fitting 63, snaps into circular snap receiving connection 65 on frame 51 according to direction arrow E. As such, frame 51 slightly bends and contracts to receive fitting 63.

Frame 51 also facilitates emptying of bag 50 by providing a more stable handle 53 than would otherwise be available using collapsible bag 50 alone. Support bar 69 provides added support to upper arch segments 71 and 73.

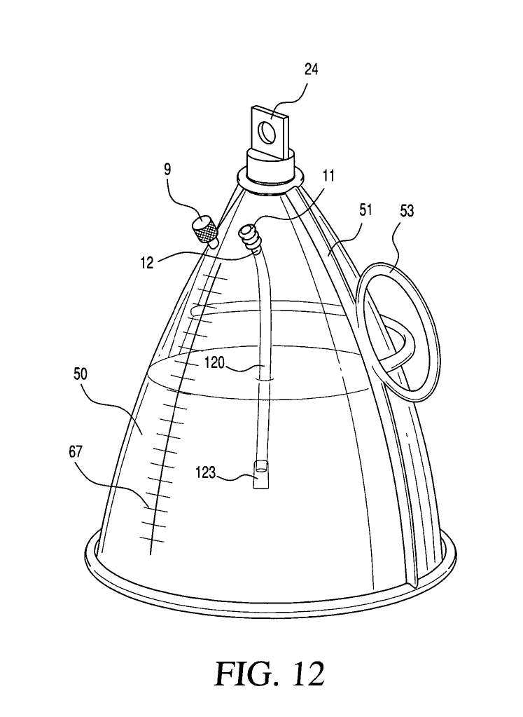

Referring to FIG. 12, once snapped into position, bag 50 experiences appropriate vertical tension from connection 65, and radially-directed horizontal tension from bottom 61, to allow for repeatable, accurate volumetric urine measuring according to printed or embossed indicia 67. Bag 50 is hung from tab 24.

Frame 51 is made from rigid plastic, but it can be made from any synthetic or natural rigid material suitable for receiving and providing adequate vertical tension to bag 50.

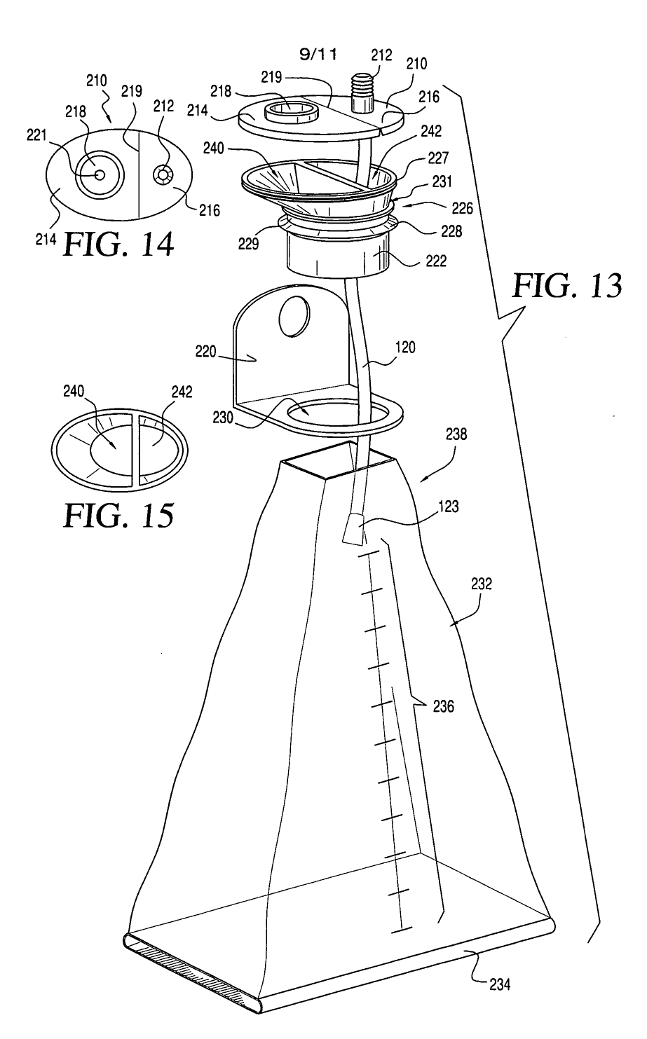

Referring to FIG. 13, in another embodiment, plastic bag assembly 238 comprises hang tab fitting 220; flexible, transparent, non-self-supporting plastic bag 232; and split cap 210, each of which attaches to hard plastic fitting 226. Hard plastic hang tab fitting 220 fits circumferentially around bottom cylinder portion 222 of fitting 226. When bearing the weight of bag assembly 238, tab 220 abuts against collar ring 228. Bag 232 is heat welded onto, and circumferentially around, bottom portion 222 of fitting 226. Bag 232 directly receives urine from fitting 226, and directly expels urine out of fitting 226. Split cap 210 is removably snapped closed onto snap ring 227 of fitting 226.

Fitting 226 snaps into a tensioning frame, which is described in greater detail below, at locking channel 229. Locking channel 229 is bounded below by wider, collar ring 228, and bounded above by wider, chin ring 231. Once installed into a frame, fitting 226 forms a conduit for urine flow into and out of bag 232.

Urine flows into bag 232 through barb inlet fitting 212, which is heat sealed onto cap 210. Urine then flows through fill tube 120, which is inserted through inlet hole 242. Tube 120 fills bag 232 from the bottom of bag 232. Fill tube 120 has one-way flutter valve 123 to prevent urine back flow to the patient.

Indicia 236 are used to volumetrically measure the urine concurrent with, and after, the filling of bag 232.

Referring to FIG. 14, vent insert 218, which has hydrophilic material impregnated with activated charcoal 221, keeps urine from either wetting or impregnating vent 218. Air that is displaced by urine as bag 232 fills with urine is therefore odorless.

Referring to FIG. 15, when bag 232 is full of urine, bag 232 is taken to an appropriate urine disposal area where lid 214 of cap 210 is opened by snapping it off of snap ring 227 and tilted open about living hinge 219. Opposing lid 216 remains snapped onto ring 227, and urine is poured out of bag 232 through pour spout 240.

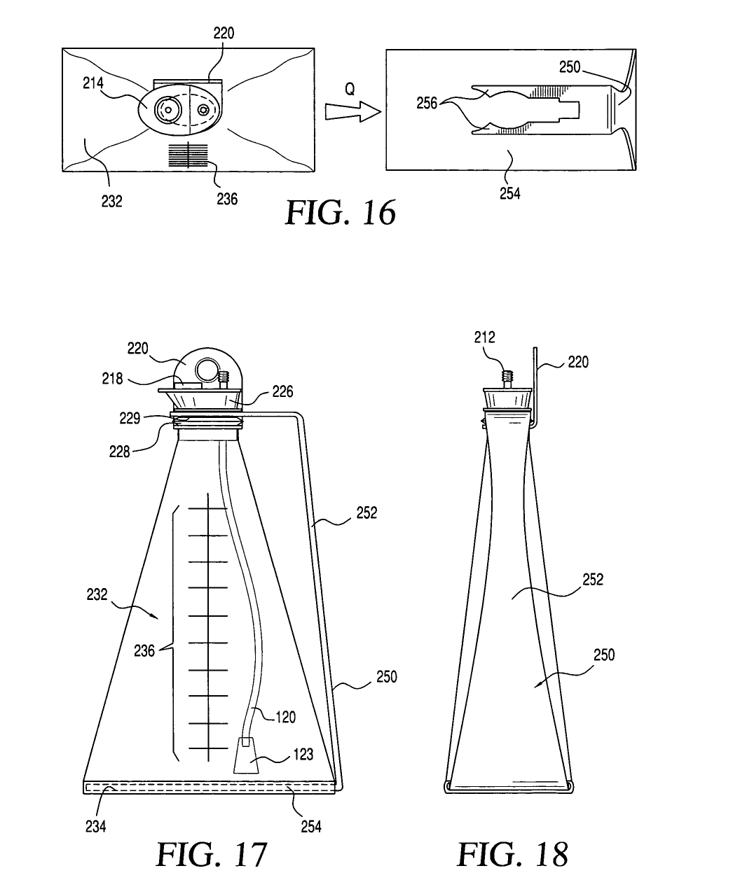

Referring to FIG. 16, once bag assembly 238 is assembled, it slides onto frame 250 according to direction arrow Q.

Referring to FIG. 17, bag 232 has additional loop of plastic 234, which has been heat sealed onto the bottom of bag 232. Loop sleeve 234 slides horizontally taught onto base 254 of frame 250. Locking channel 229 of fitting 226 then slides into place between receiving arms 256 on vertical support member 252.

Referring to FIG. 18, rigid, vertical support member 252 thus keeps bag 232 precisely tensioned between base 254 and arms 256 so that flexible non-self-supporting bag 232 is fully deployed. Once placed into this position, bag 232 experiences appropriate vertical tension from arms 256, and horizontally-directed tension from base 254, to allow for repeatable, accurate volumetric urine measuring according to printed or embossed indicia 236. When tensioned as such the resulting full and reproducible deployment of bag 232 provides accurate and precise volumetric reading without the use of additional volumetric reading instruments.

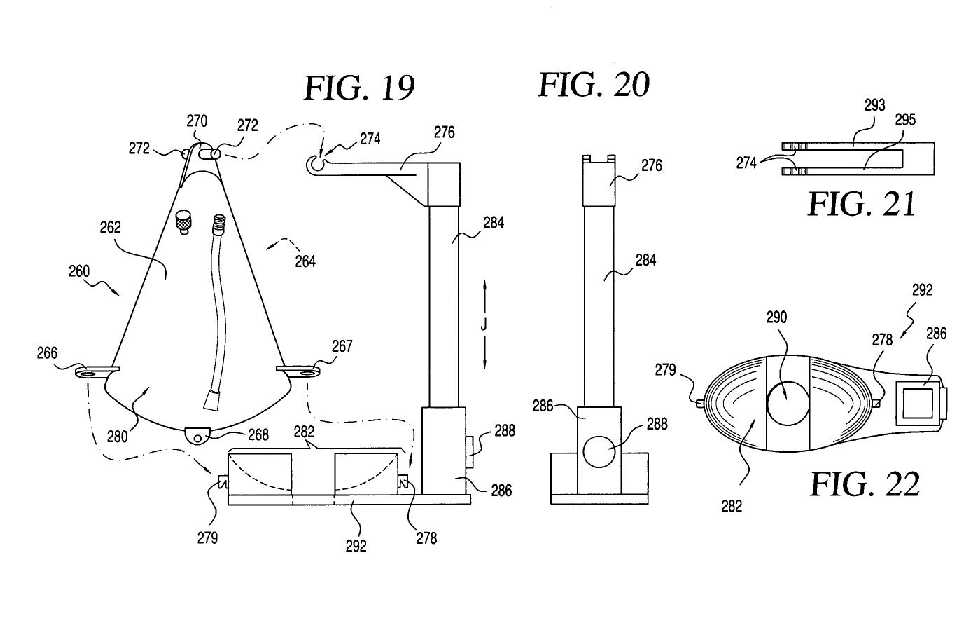

Referring to FIG. 19, in one embodiment bag 260 fits within tensioning frame 292. Bag 260 fits onto base 286 of frame 292 by its integrated tabs 266 and 267 at respective spring loaded catches 279 and 278. At its top, bag 260 fits onto telescoping arm 284 of frame 292 by its protruding bar 272 at arm 284's parallel hooks 274.

Before filling bag 260, tab 268 at the bottom of bag 260 is pulled through hole 290 so as to maximize the surface area of the bottom of bag 260 that contacts the inside surface of cradle 282. This more fully deploys the rounded bottom of bag 260.

Bag 260 is made from front 262 and back 264 flexible, plastic panels, which are heat welded together to form (1) top tab 270, through which bar 272 fits; (2) bottom tabs 266 and 267, which attach to catches 279 and 278; and (3) pull tab 268, which is used to increase bag-to-cradle surface contact area in order to more fully deploy bag 260.

Referring to FIG. 20, once attached to frame 292, spring-loaded release button 288 allows the user to telescopically raise and lower arm 284 in accordance with direction arrow J, and thereby tension bag 260.

Referring to FIG. 21, arm 284 has parallel receiving hooks 274, which extend on either side from fingers 293 and 295. Either one or both of hooks 274 can be used, but their tandem use tends to displace the tension along a wider, horizontal area of bag 260 than if exerted from a single raised tensioning point.

In another embodiment, a 4×4, 6×6, or 9×9 array of raised tensioning points are used by employing various means, including multiple hooks, loop sleeves, hook and loop fasteners, or other raised, multiple point, attachment areas. Myriad devices to effectively and easily deploy a light weight non-self-supporting, disposable urine bag are within the scope of this embodiment.

Referring to FIG. 22, cradle 282 in base 286 receives the full surface area of the bottom of bag 260. The telescopic extension of arm 284 with respect to base 286 provides appropriate tension to provide an accurate and precise volumetric measurement of urine inside bag 260. Regularly-spaced notches along the vertical length of arm 284 receive a spring loaded pin (not shown) that can reproducibly be used with multiple bags.

In an alternate embodiment, where different bags 260 vary from one to another or from multiple uses of a single bag 260, a seamless continuum of tensioning adjustment length settings can be made by the vertical adjustment of arm 284 with respect to base 286. In one such case, the tension exerted upon bag 260 is measured by a spring loaded force-measurement scale in base 286 such that the distance of telescopic tensioning is not kept constant in every instance. Rather, merely the tension force exerted on bag 260 is held constant. This way, variations such as bag material, bag stiffness, and progressive stretching of bag 262 can be accounted for nevertheless to provide accurate and reproducible volumetric measurements from bag-to-bag, and between repeated uses of the same bag. Any suitable technique based on applying the same tension force on bag 260, and not the distance bag 260 is stretched, can be used, however.

In one embodiment, bag 262 is impregnated with magnetic particles, or is otherwise magnetized, and frame 292 includes a magnetic enclosure, which is quickly and easily shut around the horizontal circumference of bag 262. The magnetic interaction between the interior of the enclosure and the magnetized bag causes bag 262 to expand for accurate and precise volumetric measurement of urine.

In one embodiment, bag 262 is electrostatically charged and frame 292 is electrostatically coupled to bag 262, which causes bag 262 to expand for accurate and precise volumetric measurement of urine.

A second aspect of the present invention is directed to a free-standing disposable urine collection bag system, which is substantially described above.

While it is apparent that the illustrative embodiments of the invention disclosed herein fulfill the objectives of the present invention, it is appreciated that numerous modifications and other embodiments may be devised by those skilled in the art. Additionally, feature(s) and/or element(s) from any embodiment may be used singly or in combination with other embodiment(s). Therefore, it will be understood that the appended claims are intended to cover all such modifications and embodiments that would come within the spirit and scope of the present invention.

I claim:

1. A self-standing urine collection bag, comprising:
a pair of opposing, substantially flat, flexible side panels that are bonded to one-another to form an enclosed body, and that move apart when the urine bag is filled with gas or urine; and
an inwardly-recessed, gusseted bottom panel that is folded when the urine bag is empty, and unfolded when the urine bag is full;
wherein the bottom panel is bonded within the body to form an air and liquid-tight closure; and
wherein the bottom panel provides support for the side panels so that, when filled with urine, the urine bag is self-supporting and can maintain a stable vertical standing position when resting on a substantially horizontal surface.

2. The urine collection bag of claim 1 wherein the bottom panel is fully deployed by a force internal to the bag, which provides accurate and precise volumetric readings of urine while urine is in the bag.

3. The urine collection bag of claim 2 wherein the bag has volumetric measuring indicia on at least one of the side panels.

4. The urine collection bag of claim 2 wherein the body is a uniform structure which includes an integrated pour handle and integrated hanging loop.

5. The urine collection bag of claim 2 wherein the deployment is maintained by a rigid external insert that operably fits into a recess in the bag.

6. The urine collection bag of claim 5 wherein the insert is a side panel separator that comprises hard foam, a rigid frame, or plastic.

7. The urine collection bag of claim 2 wherein the bag has an elastically expanding, collapsible insert that separates the side panels and deploys the gusseted bottom when the bag is empty of liquid.

8. The urine collection bag of claim 7 wherein the insert collapses during vacuum packaging of the bag so that the bag flattens, and expands when the bag is unpacked or opened for use.

9. The urine collection bag of claim 8 wherein the insert is made from reticulated foam or a rigid plastic frame.

10. The urine collection bag of claim 2 wherein the bottom panel is fully and operatively deployed by positive gas pressure internal to the bag that is greater than an ambient pressure exerted on the outside of the bag.

11. The urine collection bag of claim 10 wherein the bag is deployed by manual inflation through a blow tube inlet, or by a release of gas from a separate, compressed gas chamber.

12. The urine bag of claim 2 wherein the bag has dual-purpose, liquid inlet and outlet fittings that allow air to escape when urine enters the bag, and allow air to enter when urine exits the bag.

13. The urine bag of claim 12 wherein at least one of the fittings includes activated carbon to adsorb odors from air vented out of the bag during its filling with urine.

14. The urine bag of claim 12 wherein the bag has a handle that is made from at least one of the side panels.

15. A self-standing urine collection bag, comprising:
a bag of flexible material having a top and a bottom portion, the outer surfaces of which are operatively and releasably coupled to respective upper and lower ends of a rigid external frame;
wherein the rigid frame provides support for the bag so that, when filled with urine, the bag can maintain a stable vertical standing position when resting on a substantially horizontal surface.

16. The urine collection bag of claim 14 wherein the frame operably places the bag in tension across multiple internal distances so that a measurement of volume of urine in the bag, using measuring indicia on the bag or frame, substantially and reproducibly conforms to an accurate volume of urine.

17. The urine bag of claim 15 wherein the tension placed upon the bag is three-1 dimensional.

18. The urine bag of claim 16 wherein the frame has a telescoping arm, the adjustable movement of which imparts the tension.

19. The urine collection bag of claim 17 wherein the bag has volumetric measuring indicia printed or embossed on one of the side panels.

20. The urine collection bag of claim 16 wherein the bag operatively has a handle that is made from the rigid frame.

21. A self-standing urine collection bag system, comprising:
a flexible, side portion that is formed of one or more plastic sheet panels bonded to one another along their sides and tops to form a body that expands when the urine collection bag is filled;
an inwardly-recessed, bottom panel or plug that is bonded to an internal wall of the side portion to form a supportive interface;
wherein the supportive interface provides support for the side portion so that, when filled with urine, the urine collection bag is self-supporting and can maintain a stable vertical standing position when resting on a substantially horizontal surface;
wherein the supportive interface provides regular and full deployment of the bag for accurate measurement of urine while urine is in the bag, by measuring indicia on the bag.

22. The urine collection bag system of claim 21 wherein the bag has dual purpose liquid inlet and outlet fittings that allow air to escape when urine enters the bag, and allow air to enter when urine exits the bag.

23. The urine bag system of claim 22, further comprising a frame that operably places the bag in tension across multiple internal distances so that a measurement of volume of urine in the bag, using measuring indicia on the bag, substantially and reproducibly conforms to an accurate volume of urine.

24. The bag system of claim 22 wherein at least one of the fittings includes activated carbon to adsorb odors from air vented out of the bag during filling with urine.

25. The urine collection bag system of claim 21 wherein the bag has a handle that is made from a cylindrical side portion.

26. The urine collection bag system of claim 24 wherein the bag is made from a cylindrical body, and at least one embossing structure on the side portion provides vertical support for the bag.

27. The urine collection bag system of claim 24 wherein a rod runs from the top of the cylindrical portion to the bottom panel or plug to provide additional vertical support, and stability while emptying the bag.

28. The urine bag system of claim 22 wherein the inlet and outlet fittings are sealed between the two sheet panels.
